(19) United States
(12) Patent Application Publication
(10) Pub. No.: US 2009/0280501 A1
(43) Pub. Date: Nov. 12, 2009

(54) A Method And A Kit For Diagnosing Type 2 Diabetes, Metabolic Syndrome, Sub Clinical Atherosclerosis, Myocardial Infarct, Stroke Or Clinical Manifestations Of Diabetes

(75) Inventors: German Camejo, Molndal (SE); Pia Davidsson, Molndal (SE); Johannes Hulthe, Molndal (SE)

Correspondence Address: Pepper Hamilton LLP, 400 Berwyn Park, 899 Cassatt Road, Berwyn, PA, 19312-1183, US

(73) Assignee: ASTRAZENECA AB, Sodertalje (SE)

(21) Appl. No.: 11/911,118

(22) PCT Filed: Apr. 10, 2006
(86) PCT No.: PCT/SE2006/000425

§ 371 Date: Oct. 10, 2007

(30) Foreign Application Priority Data
Apr. 11, 2005 (SE) 0500808-1

(51) Int. Cl. G01N 33/53

(52) U.S. Cl. 435/7.1

(57) ABSTRACT

A method and a kit for diagnosing or prognosing susceptibility to develop type 2 diabetes, the metabolic syndrome, sub clinical atherosclerosis, myocardial infarct, stroke or clinical manifestations of diabetes in a subject. The method comprises detecting and quantifying the amount of bound protein (s), (apoCIII, apoCI, apoA1 or apoE) on small dense low density lipoproteins (sdLDL) particles in a blood sample from said subject.

### TECHNICAL FIELD

The present invention relates to a method for diagnosing or prognosing susceptibility to develop type 2 diabetes, the metabolic syndrome, sub clinical atherosclerosis, myocardial infarct, stroke or clinical manifestations of diabetes in a subject.

### BACKGROUND OF THE INVENTION

The entrapment and modifications of LDL (low density lipoprotein) particles in the proteoglycan layer of the intima appears to be critical initial steps in atherogenesis. Most of the interactions of LDL with other macromolecules and cells, including those that contribute to its atherogenicity, are controlled by segments of the non-exchangeable apoB100 exposed at the particle surface. (Chan L, J Biol Chem 1992; 267:25621-25624; Camejo G, et al, Pathological significance 1998; 139:205-222). Exchangeable apolipoproteins and proteins seem to be able to modulate some of these interactions when adsorbed at the LDL surface. The operationally defined lipoprotein class with densities 1.019-1.063 is a collection of particles that differ in size, lipid and apolipoprotein composition (Alaupovioc P, Methods Enzymol 1996; 263:32-60). Elevated levels of the small dense LDL (sdLDL) subclass are strongly associated to coronary disease progression and this has been proposed as a marker of the atherogenic dyslipidemia of insulin resistance and type 2 diabetes (Krauss R M, World Rev Nutr Diet 1997; 80.22-43; Taskinen M R, Diabetologia, 2003; 46:733-749). Furthermore, prevalence of sdLDL is high in subjects with preclinical femoral and carotid atherosclerosis (Hulthe J, et al, Arteriosclerosis, Thrombosis and Vascular Biology 2000; 20:2140-2147). Several hypotheses have been proposed to explain the atherogenicity of sdLDL. These particles have in vitro increased affinity for arterial proteoglycans (PGs) and once associated with them become more easily modifiable by enzymatic and oxidative processes than larger more buoyant LDL (Camejo G, et al, Pathological significance 1998; 139:205-222; Hurt-Camejo E, et al, Journal of Lipid Research 1990; 31:1387-1398).

From buoyant LDL (large) to denser LDLs (small) there is a gradual change in lipid composition and surface properties of the particles. The decrease in phospholipids and free cholesterol in the particle surface is associated with changes in the exposure of segments of the apoB-100 that modify its affinity for the apoB/E receptor. Such changes can also explain partially the larger affinity of sdLDL for proteoglycans since sequences 3359-3369 (B-site) and 3145-3157 (A-site) that participate in proteoglycan binding also participate in receptor-binding (Chan L, J Biol Chem 1992; 267:25621-25624). This has led to the hypothesis that sdLDL has higher affinity for PGs because these positive sequences in the surface become closer and more exposed (Camejo G, et al, Pathological significance 1998; 139:205-222; Hurt-Camejo E, et al, Journal of Lipid Research 1990; 31:1387-1398).

High apolipoprotein CIII (apoCIII) content in LDL is receiving much attention since subjects with this phenotype are at much higher risk of cardiovascular events than those with a low apoCIII content in LDL, independently of LDL-cholesterol values (Lee S-J, et al, Arterioscler Thromb Vasc Biol 2003; 23(5):853-858; Lee S-J, et al., Am J Cardiol 2003; 92(2):121-124). ApoCIII, by inhibiting hydrolysis of triglycerides in very low density lipoprotein (VLDL), causes hypertriglyceridemia but it is not clear why it should increase the atherogenicity of LDL. Recently it was shown that when associated to LDL apoCIII increases the lipoprotein affinity for arterial proteoglycans (Olin-Lewis K, et al, J Lipid Research 2002; 43(11):1969-1977).

WO 2004/085996 discloses a method of using the sizes and levels of high density HDL and LDL from plasma from a subject in order to determining said subjects likelihood of developing a cardiovascular- metabolic- or age-related disease.

### SUMMARY OF THE INVENTION

The object of the present invention is to easily be able to determine or predict the likelihood of developing type 2 diabetes, the metabolic syndrome, sub clinical atherosclerosis, myocardial infarct, stroke or clinical manifestations of diabetes in a subject.

This object has been solved in that a method is provided for diagnosing or prognosing susceptibility to develop type 2 diabetes, the metabolic syndrome, sub clinical atherosclerosis, myocardial infarct, stroke or clinical manifestations of diabetes in a subject, comprising detecting and quantifying the amount of bound protein(s) on small dense low density lipoproteins (sdLDL) particles in a blood sample from said subject.

According to another aspect of the invention, there is provided, the use of a method for profiling the type and amount of protein(s) bound on sdLDL particles as a biomarker for selecting patients for clinical trials.

According to yet another aspect of the invention, there is provided, the use of a method for profiling the type and amount of protein(s) bound on sdLDL particles in a subject as a biomarker for the evaluation of the results of pharmacological intervention of said subject.

According to a further aspect of the invention, there is provided, a diagnostic kit for diagnosing or prognosing susceptibility to develop type 2 diabetes, the metabolic syndrome, sub clinical atherosclerosis, myocardial infarct, stroke or clinical manifestations of diabetes in a subject, comprising a protein chip capable of detecting and quantifying proteins bound on sdLDL particles.

According to another aspect of the invention, there is provided, a method to predict arterial intima media thickness, comprising detecting and quantifying the amount of bound protein(s) on small dense low density lipoproteins (sdLDL) particles in a blood sample from said subject.

The methods according the present invention can be used to identify approximately 80% of the patients with type 2 diabetes without further information. The described specific profile of proteins associated with small dense LDL from patients with type 2 diabetes provide additional information about biochemical characteristic of the atherogenetic LDL associated with this disease beyond that provided by size evaluation alone. These biomarkers should be useful for patient selection in phase I to III studies in patients with dyslipidemia/diabetes/atherosclerosis, and also for the evaluation of the results of pharmacological intervention.

### BRIEF DESCRIPTION OF THE FIGURES

FIG. 1. ApoB100 profiles of serum LDL separated in a preformed gradient of buffers containing 140 mM NaCl, in mixtures of D₂O and H₂O. Profile A is from healthy control subject. Profile B from a patient with type 2 diabetes and the LDL B phenotype.

FIG. 2. SELDI-TOF-MS protein profiles of sdLDL from 10 out of 23 controls and 10 out of 22 patients with diabetes (study 2) analysed on Q10 protein chip arrays.

FIG. 3. SELDI-TOF-MS protein profiles of sdLDL from 10 out of 23 controls and 10 out of 22 patients with diabetes (study 2) analysed on CM10 protein chip arrays.

FIG. 4a and FIG. 4b. Individual values of a/ apoCIII/apoB and b/ apoCI/apoB in buoyant (Fr4) compared with (Fr5) sdLDL particles in controls (subjects 1-23) and patients (subjects 24-45).

FIG. 5. Comparison of SELDI-TOF-MS protein profiles on sdLDL after deuterium fractionation and on PG-LDL complexes respectively from 1 out of 23 controls and 1 out of 22 patients with diabetes (study 2) analysed on Q10 protein chip arrays. The three bands with molecular masses of 8920, 9420 and 9720 Da respectively were increased in sdLDL in patients compared to controls (p<0.001) as well as in PG-LDL complexes in patients than that of controls (p<0.001).

FIG. 6. Correlation between the total content of apoCIII in the sdLDL of patients with type 2 diabetes and the amount of LDL cholesterol insolubilized by the association with aortic versican in vitro.

### DETAILED DESCRIPTION OF THE INVENTION

The present invention provides a method for diagnosing or prognosing susceptibility to develop type 2 diabetes, the metabolic syndrome, sub clinical atherosclerosis, myocardial infarct, stroke or clinical manifestations of diabetes in a subject, comprising detecting and quantifying the amount of bound protein(s) on small dense low density lipoproteins (sdLDL) particles in a blood sample from said subject.

Preferably, said method is performed on serum separated from said blood sample. Optionally, a fraction containing sdLDL particles is first separated from the serum before the detection and quantification of the amount of bound protein(s).

The amount of bound protein(s) on small dense low density lipoproteins (sdLDL) particles is compared to quantified amounts from healthy control subjects. This could be carried out on control subjects at the same time as the test subjects, or it could be historical control data.

In one embodiment, a method is provided for diagnosis of type 2 diabetes or prediction of acquiring type 2 diabetes, diagnosis of the metabolic syndrome, sub clinical atherosclerosis, clinical manifestations of diabetes or clinical manifestations of atherosclerosis (i.e. myocardial infarct and stroke) in a subject, comprising the steps of:

a) taking a blood sample from said subject and collect the serum of said sample,
b) separation the fraction containing small dense low density lipoproteins (sdLDL particles),
c) detect and quantify the level of bound protein(s) on the separated sdLDL particles obtained from step b),
d) identifying said protein(s), and
e) compare to levels from healthy control subjects.

The blood sample could be taken just before the above measurement, but it could also be a blood sample that has been taken from the subject in question a long time before and have been stored in an appropriate way prior to the above measurement.

The separation of the fraction containing small dense low density lipoproteins (sdLDL particles) could be performed using any technique known to the skilled person for separation of sdLDL particles from the other components of blood. It could for example be performed by running a density gradient ultra centrifugation with suitable buffers such as those prepared in D₂O or by precipitation of the sdLDL particles from serum with an arterial proteoglycan solution. It could also be

The detection can be performed using 1D gel electrophoresis. The detection and quantification can be performed by immunoassay. Preferably, the detection and quantification of the amounts of bound protein(s) on sdLDL particles is performed by means of Surface Enhanced Laser Desorption Ionization (SELDI) analysis.

The term “bound” as used herein is meant to be interpreted to include proteins bound to the sdLDL particle in any kind of non-covalent manner, such as hydrogen bonding, hydrophobic interaction, van der Waal forces, ionic interaction. The proteins are retained on the surface of the lipoprotein particles with different affinity and can be in equilibrium with a free pool or with those associated with other lipoprotein classes.

The bound protein to the sdLDL particles can be one or more selected from the group consisting of apolipoprotein CIII (apoCIII), apolipoprotein CI (apoCI), apolipoprotein (apoAI) or apolipoprotein (apoE), wherein apoCIII occurs as three isoforms and apoCI occurs as two isoforms (Pullinger et al., 1997).

For some of the bound proteins to the sdLDL particles as estimated in the above method, an elevated amount of one or more bound proteins to sdLDL particles relative to that in control subjects is indicative of whether the subject is suffering from and/or being at risk for developing diabetes type 2, the metabolic syndrome, sub clinical atherosclerosis, clinical manifestations of diabetes or clinical manifestations of atherosclerosis (i.e. myocardial infarct and stroke). For instance, the level of apoCIII increases as compared to healthy subjects.

For some of the bound proteins to the sdLDL particles as estimated in the above method, a lowered amount of one or more bound proteins to sdLDL particles relative to that in control subjects is indicative of whether the subject is suffering from and/or being at risk for developing diabetes type 2, the metabolic syndrome, sub clinical atherosclerosis, clinical manifestations of diabetes or clinical manifestations of atherosclerosis (i.e. myocardial infarct and stroke). For instance, the amounts of apoCI, apoAI and apoE decrease as compared to healthy subjects.

In one embodiment the combination of apoCIII and apoCI bound to sdLDL particles is measured and reveals that an elevated amount of one or more bound proteins to sdLDL particles relative to that in control subjects is indicative of whether the subject is suffering from and/or at risk for developing diabetes type 2 the metabolic syndrome, sub clinical atherosclerosis, clinical manifestations of diabetes or clinical manifestations of atherosclerosis (i.e. myocardial infarct and stroke).

In another embodiment the combination of apoCIII, and/or apoAI and/or apoE and/or apoCI bound to sdLDL particles is measured and reveals that the amount of apoCI and/or apoAI and/or apoE bound to sdLDL particles is lowered and the amount of apoCIII bound to sdLDL particles is elevated in subjects having and/or at risk for developing type 2 diabetes, the metabolic syndrome, sub clinical atherosclerosis, clinical manifestations of diabetes or clinical manifestiations of atherosclerosis (i.e. myocardial infarct and stroke), as compared to healthy control subjects.

By using the methods according to the invention, the detection and quantification of apoCIII, apoCI, apoA1, apoE or a combination of these proteins bound to sdLDL particles can be used as biomarker/biomarkers for selecting patients for clinical trials.

A biomarker can be defined as a measurable and evaluable indicator of a normal biologic process, pathogenic process or as a pharmacological response to a therapeutic intervention e.g., by administration of a therapeutic agent. Hence, the estimated levels of each of said bound proteins or a combination of said proteins to sdLDL particles, will function as a biomarker in patients with dyslipidemia, and/or diabetes and/or atherosclerosis and therefore provides an excellent tool for the selection of suitable subjects for Phase I to III studies. The estimated levels of the above proteins bound to sdLDL particles can also be used for selecting patients for the evaluation of the results of pharmacological intervention of said subject.

Hence, the use of a method for profiling the type and amount of protein(s) bound on sdLDL particles in a subject can be used as a biomarker for selecting patients for clinical trials or for the evaluation of the results of pharmacological intervention of said subject. A combination of apoCI and apoCIII can be used. In one embodiment, the amount of apoCI bound to sdLDL particles is decreased and the amount of apoCIII bound to sdLDL particles is increased as compared to healthy control subjects. Further, a combination of apoCI, and/or apoA1, and/or apoE and/or apoCIII can be used. In one embodiment, the amount of apoCI, apoA1 and apoE bound to sdLDL particles is decreased and the amount of apoCIII bound to sdLDL particles is increased as compared to healthy control subjects.

The invention also relates to a diagnostic kit for diagnosing or prognosing susceptibility to develop type 2 diabetes, the metabolic syndrome, sub clinical atherosclerosis, myocardial infarct, stroke or clinical manifestations of diabetes in a subject, comprising a protein chip capable of detecting and quantifying proteins bound on sdLDL particles. The chip can be provided with coupled arterial proteoglycans. The chip could also be used for direct determination of sdLDL bound proteins. The kit will provide an easy and a rapid way to carry out the methods of the invention in order to get information from a blood sample regarding the status of the subject.

The invention also relates to a method to predict arterial intima media thickness in a subject (see example 8), comprising detecting and quantifying the amount of bound protein(s) on small dense low density lipoproteins (sdLDL) particles in a blood sample from said subject.

The plasma or serum lipoproteins with densities between 1.019 g/ml and 1.063 g/ml have been defined operationally as low density lipoproteins (LDL) using ultracentrifugal procedures (de Lalla, O., and J. Gofman, 1954, p. 459-478, in D. Glick ed., Methods in Biochemical Analysis, vol. 1. Wiley, Interscience, New York). The LDL can be subfractionated in overlapping subclasses of increasing density within the 1.019-1.063 g/ml range. There is a strong correlation between increasing density and decreasing size evaluated by electrophoresis, light scattering, electron microscopy or nuclear magnetic resonance (Krauss, R. M., 2004, Diabetes Care 27:1496-1504). Frequently, LDL particles with density between 1.019-1.030 g/ml are considered large and buoyant whereas those with densities 1.030-1.063 g/ml are designated as small and dense. In the present study a proteomic approach was used to compare the exchangeable apolipoproteins associated with small dense and large LDL particles in healthy controls and two types of patients with the B phenotype. One group of patients had sub clinical evidence of peripheral atherosclerosis and many of the characteristics of the metabolic syndrome. A second group studied had type 2 diabetes. The proteomic evaluation was applied to LDL subclasses isolated by ultra centrifugation in D₂O density gradients with physiological salt concentration. The proteomic method used involved profiling of bound proteins to the LDL subclasses using surface enhanced laser desorption/ionization time-of-flight mass spectrometry (SELDI TOF MS) and subsequent identification of altered proteins by mass spectrometry (MS) and immunoblotting. The results obtained identified the apolipoproteins apoCIII, apoCI, ApoAI and apoE being bound to LDL density subclasses.

The objective of this study was to explore whether buoyant and dense subclasses of LDL in patients with sub clinical atherosclerosis and the LDL phenotype B and patients with type 2 diabetes and the phenotype B have an specific pattern of bound apolipoproteins that could be different from that of matched healthy controls. The patients in the first study, besides having a LDL B phenotype, had a significant wider waist, higher fasting insulin, higher fasting blood glucose, higher triglycerides and lower HDL cholesterol than the controls (Table 1). Thus they shared several components of the metabolic syndrome. The metabolic syndrome can be defined either according to Grundy, et al., 2004, Arterioscler Thromb Vase Biol, 24:13e-18 orIsomaa, B., et al., 2001, Diabetes Care 24:683-9.

All the patients in this study showed small or moderate carotid atherosclerosis and 3 of them also presented femoral plaques. None of the matched controls showed the B phenotype (Table 1). The patients with type 2 diabetes in the second study, besides the B phenotype, had also wider waist, lower HDL cholesterol, higher triglycerides and much higher prevalence of carotid and femoral plaques than the matched controls (Table 2).

To analyse the exchangeable apolipoproteins bound to the two LDL subclasses an ultra centrifugal procedure that maintained the lipopoproteins at physiological salt concentration and pH was used. This procedure minimizes the possibilities of altering the distribution of exchangeable proteins and apolipoproteins bound to the LDL particles in circulation, as t occurs with ultra centrifugation at high salt concentrations (MacConathy W M, Koren K, Wieland H et al, Journal of Chromatography 1985; 342:47-66). In addition, a proteomic procedure including profiling of bound proteins of the two LDL subclasses by SELDI analysis and subsequent identification of the altered protein peaks with MS and immunoblotting were performed. This approach allowed quantitative evaluation and identification of several apolipoproteins with an acceptable coefficient of variation (14-24%). The results show that the sdLDL subclass with density 1.040-1.060±0.005 g/ml in patients with sub clinical peripheral atherosclerosis, with the B phenotype and patients with type 2 diabetes and the B phenotype are enriched in apoCIII's (three isoforms) and depleted of apoCI's (two isoforms), apoAI and apoE when compared with the equivalent fraction of healthy controls. Furthermore, the pattern of a sdLDL particle with high apoCIII and low apoCI allowed to identify 80% of the patients studied without need of any other supplementary information about anthropometric or clinical chemistry parameters.

Total plasma apoCIII and that associated with apoB100-containing particles are strong predictors of coronary risk, especially in women and men affected by the metabolic syndrome (Onat A, et al, Atherosclerosis 2003/5 2003; 168(1):81-89). Furthermore, in patients with type 2 diabetes and coronary disease the quartile of LDL with the highest apoCIII content increases the relative risk of new coronary events more than 6 fold relative to the quartile with the lowest apoCIII content (Lee S-J, et al, Arterioscler Thromb Vasc Biol 2003; 23(5):853-858). Our results indicate that in patients with markers of the metabolic syndrome and with type 2 diabetes the atherogenic sdLDL is specially rich in apoCIII and impoverished in apoCI, apoAI and apoE.

Small and dense LDL subclasses are the main catabolic product of large, triglyceride-rich VLDL and recent evidence indicates that its apoCIII content is related to its rate of production (Marcoux C, et al, Metabolism 2001; 50(1):112-119). In addition, kinetics studies in visceral obese subjects showed that a high content of apoCIII in triglyceride-rich VLDL is related to its combined overproduction and decreased catabolism (Chan D C, et al, Metabolism 2002; 51(8):1041-1046). These results suggest a mechanism by which apoCIII-enriched dense LDL can be generated in the patients with the metabolic syndrome and type 2 diabetes that have been studied. However, there is no explanation for the reduced content of apoCI, apoAI and apoE that was observed in the dense LDL subclass of such patients in the present study. One possibility is that this reflects the reduced availability of these apolipoproteins in the plasma of insulin resistant patients and a reduced capacity of the small, dense LDL, apoCIII-rich particle surface to retain them as has been shown for apoE in large VLDL (Breyer E D, et al, J Lipid Research 1999; 40(10):1875-1882). However, along the same line of reasoning one could speculate that in the dense LDL of healthy controls the high content apolipoproteins, i.e., apoAI, apoE and apoCI displaces the apoCIII from the particle LDL surface.

It is still not clear why sdLDL is more atherogenic than buoyant particles but its high affinity for arterial proteoglycans could increase its retention in the intima, an important first step in atherogenesis (Camejo G, et al, Pathological significance 1998; 139:205-222). Our results suggest that insulin resistant and type 2 diabetic patients with the B phenotype have high circulating levels of sdLDL rich in apoCIII that could increase its interaction with the proteoglycans of the arterial intima and thus facilitate its in situ oxidative and enzymatic modifications. Moreover, our results show that such particles are impoverished on apoAI that have been postulated to protect LDL in the arterial wall against atherogenic modifications (Rohrer L, et al, Current Opinion on Lipidology 2004; 15(3):269-278). Based on these considerations it could be assumed that this class of LDL, common in the dyslipidemia of insulin resistance, may be one of the causes of plaque development. Such role could explain in part the striking association of increased apoCIII in LDL with the augmented risk for cardiovascular disease in insulin resistance and type 2 diabetes.

### EXPERIMENTAL SECTION

The invention will now be highlighted in more detail by means of the following non-limiting examples. In the examples, the evaluation of the differences between controls and patients were made with two-tailed Student's t-test and a p<0.05 was considered significant.

### Example 1

### Selection of Subjects

In the first study 10 subjects with sdLDL (pattern B phenotype), subclinical atherosclerosis in the carotid arteries, but without concurrent medication were compared with 10 age and sex matched healthy controls with no risk factors in the metabolic syndrome (NCEP definition, with the addition of hyperinsulinemia, as indicator of insulin resistance), total cholesterol <6.5 mmol/l, no clinical cardiovascular disease, no subclinical atherosclerosis (no plaque occurrence in the carotid or femoral arteries) and no medication and clinically healthy (Table 1).

In the second study 21 patients with type 2 diabetes randomly selected from the 74 available patients in the Atherosclerosis and Insulin Resistance study (AIR) were compared with 23 age and matched healthy controls with no risk factors of the metabolic syndrome, as defined by the NCEP guidelines and the addition of hyperinsulinemia, as indicator of insulin resistance (Table 2). All subjects were recruited from the AIR study at 3-year follow-up examination. This study has previously been very well described (Hulthe J, et al, Arteriosclerosis, Thrombosis and Vascular Biology 2000; 20:2140-2147; Sigurdardottir V, et al., Diabetes Care 2004; 27(4):880-884). The LDL phenotype of all the participants was established by gel gradient electrophoresis as previously described (Hulthe J, et al, Arteriosclerosis, Thrombosis and Vascular Biology 2000; 20:2140-2147). The Ethics Committee at Sahlgrenska University hospital approved the studies.

TABLE 1

| | Controls | Phenotype B | P-value |
|---|---|---|---|
| Waist (cm) | 87 | 97 | 0.029 |
| LDL (mmol/L) | 3.81 | 3.55 | >0.30 |
| HDL (mmol/L) | 1.44 | 0.96 | <0.001 |
| Tg (mmol/L) | 0.85 | 2.35 | <0.001 |
| Fasting blood glucose | 4.7 | 5.2 | 0.029 |
| Plasma insulin | 29.0 | 74.5 | 0.007 |
| HsCRP | 0.78 | 1.88 | 0.043 |

Carotid artery plaque

| | | |
|---|---|---|
| No | 10 | 0 |
| Small | 0 | 6 |
| Moderate/Large | 0 | 4 |

Femoral artery plaque

| | | |
|---|---|---|
| No | 10 | 7 |
| Small | 0 | 1 |
| Moderate/large | 0 | 2 |

Characteristics of subjects in study 1, 10 healthy controls and 10 patients with phenotype B and peripheral atherosclerosis.

TABLE 2

| | Controls | Phenotype B | P-value |
|---|---|---|---|
| Waist (cm) | | | <0.001 |
| LDL (mmol/L) | 3.57 | 3.40 | >0.30 |
| HDL (mmol/L) | 1.55 | 1.10 | <0.001 |
| Tg (mmol/L) | 0.87 | 2.08 | <0.001 |
| Fasting blood glucose | 4.64 | 9.2 | <0.001 |
| Plasma insulin | 65.1 | 95.6 | 0.004 |
| HsCRP | 0.78 | 1.88 | 0.043 |

Carotid artery plaque

| | | |
|---|---|---|
| No | 15 | 5 |
| Small | 0 | 6 |
| Moderate/Large | 3 | 8 |

Femoral artery plaque

| | | |
|---|---|---|
| No | 18 | 11 |
| Small | 1 | 0 |
| Moderate/large | 3 | 9 |

Characteristics of subjects in study 2, 23 healthy controls and 21 patients with type 2 diabetes, phenotype B and peripheral atherosclerosis.

### Example 2

### LDL Fractions

The LDL density subclasses were isolated from serum samples (1.0-1.35 ml) by ultra centrifugation in preformed gradients of buffers containing 140 mM NaCl, 10 mM Na₂-EDTA, Hepes 10 mM, pH 7.2 prepared with different amounts of deuterium oxide (D₂O) as previously described (Hallberg C, et al., Journal of Lipid Research 1994; 35:1-9). Fractions were collected by upward displacement of the gradient. Total protein content of LDL fractions was determined using the DC Protein Assay (Bio-Rad, Hercules, Calif., USA) according to the manufacturer's instructions with bovine serum albumin as standard. ApoB was determined in LDL fractions by a turbidimetric method using an anti-human apoB antibody (Dakopatts, Denmark). Total cholesterol was measured calorimetrically (Roche Diagnostics, RmBH, Manheim, Germany). The density of the solutions used and of the gradients after centrifugation was established by gravimetry (Hallberg C, et al., Journal of Lipid Research 1994; 35:1-9). Diameters of the LDL subclasses were evaluated by gradient gel electrophoresis essentially as described by Krauss and Burke (Krauss R M, et al., Journal of Lipid Research 1982; 23:97-104).

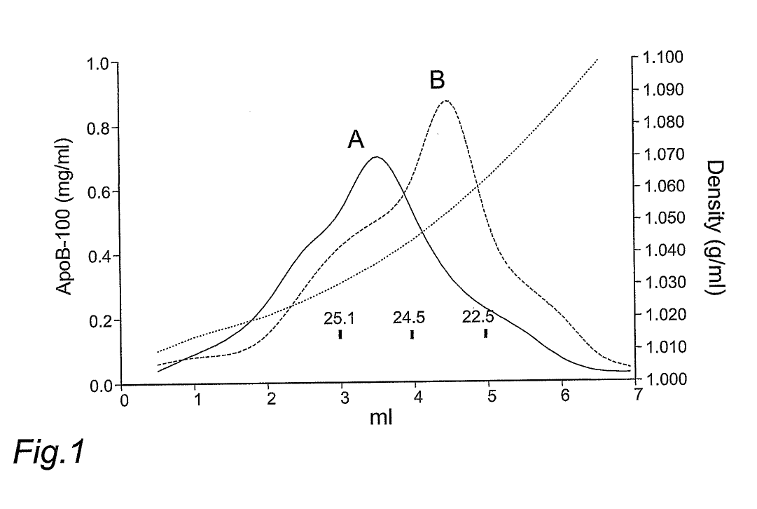

Density gradient fractionation profiles in D₂O buffers of serum from a subject with the B phenotype and a healthy control are presented in FIG. 1. The ApoB profiles were obtained collecting 0.5 ml fractions, however for the proteomic analyses fractions of 1.0 ml were used in order to maintain the subsequent analyses within manageable numbers. As indicated in FIG. 1 with this procedure fraction 4 contained LDL with densities 1.030-1.040±0.005 g/ml, the most abundant LDL subclass from all control subjects in study 1. Such fraction corresponds approximately in size range to that of the LDL2b-LDL3a range of Krauss (Krauss R M, Diabetes Care 2004; 27(6):1496-1504). Fraction 5 contained LDL with densities 1.040-1.060±0.005 g/ml that corresponds approximately to the size ranges of fractions LDL3b to 4b of Krauss (Krauss R M, Diabetes Care 2004; 27(6):1496-1504) (FIG. 1). This fraction was the most abundant class in 8 of the 10 studied subjects with the B phenotype in study 1. In the second study 20 of the 23 controls showed a maximum at fraction 4 and 19 of the 21 patients with type 2 diabetes showed a maximum at fraction 5. These results indicate that the D₂O density gradient correlates with the LDL phenotyping using the gel electrophoresis procedure and show the high prevalence of sdLDL in both types of patients (Hulthe J, et al, Arteriosclerosis, Thrombosis and Vascular Biology 2000; 20:2140-2147).

### Example 3

### SELDI Analysis of LDL Bound Proteins

In the two studies LDL fraction 4, density 1.020-1.040 g/ml and fraction 5, density 1.040-1.060 g/ml, from all subjects were analysed on two types of protein chip surfaces; cationic (CM10) and anionic (Q10) protein chips using 50 mM ammonium acetate, pH 6.0 and 50 mM Tris-HCL, pH 9.0 respectively. All samples were processed using a Biomek Laboratory workstation (Beckman-Coulter) modified to make use of a protein chip array bio processor (Ciphergen Biosystems). Twenty μl of each LDL fraction was mixed with 80 μL binding buffer and the mixture was added to the chip surfaces and incubated for 30 min. The spots were then washed three times with 100 μl binding buffer for 5 min to reduce unspecific binding and finally twice with 100 μl deionised water respectively. Two different types of matrices were used including sinapinic acid (SPA) (Aldrich Chem Co, Milw, Wis.) and α-cyano-4-OH-cinnamic acid (CHCA) (Bruker Daltonics, Germany). A saturated solution of SPA (diluted 1:2 v/v) or CHCA (diluted 1:5 v/v) with 50% acetonitrile containing 0.5% TFA were applied twice to each dried sample spots to form crystals.

The arrays were subsequently read in a protein chip reader system for SELDI analysis (PBS II, Ciphergen Biosystems). The reader was calibrated externally using the all-in-protein/peptide standards diluted in the SPA/CHCA matrix and directly applied onto a spot of the normal-phase protein chip (NP-20 protein chip array). Protein profile comparisons were performed after normalization for total ion current of all spectra collected in one experiment. Significance threshold was set to a p<0.05.

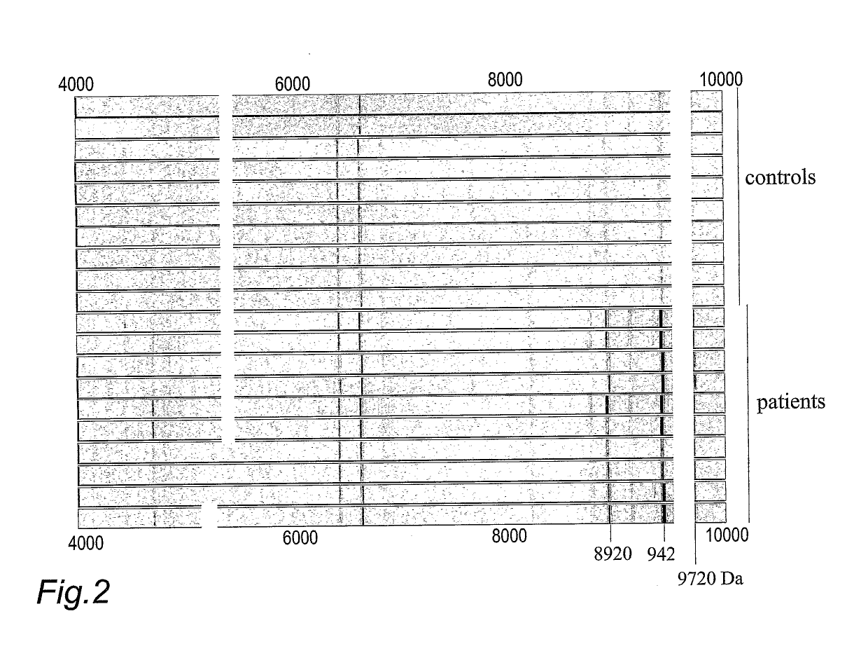

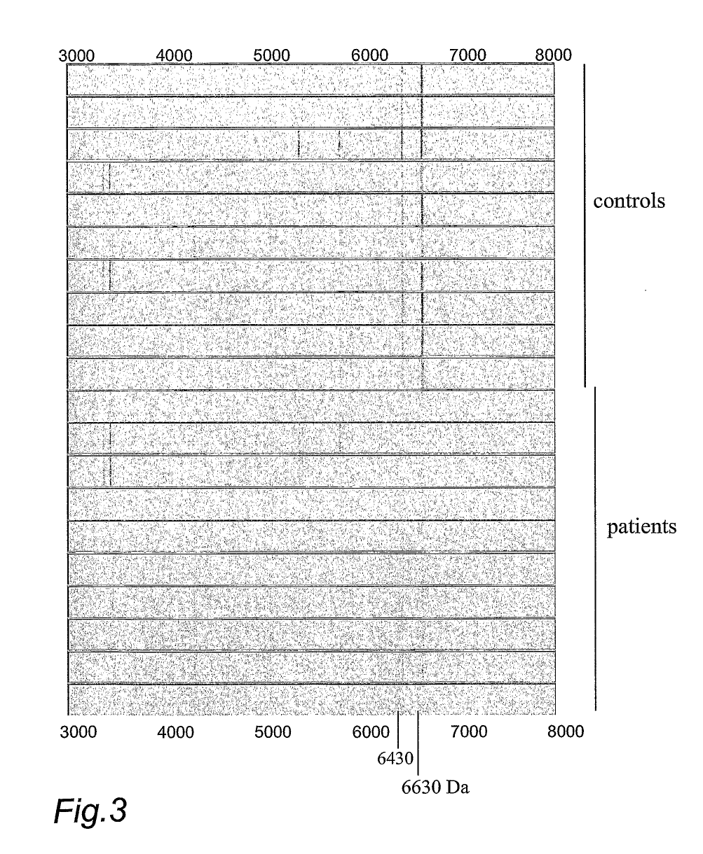

Bound proteins to the LDL subclasses were analysed on the Q10 (anionic) and CM10 (cationic) protein chip arrays by SELDI with variation coefficients between 14-24% for duplicate runs. FIG. 2 shows representative profiles in the molecular weight range between 4000-10 000 Da from the analysis of sdLDL from patients and controls in study 2 on the Q10 protein chip arrays using SELDI. Clearly higher intensity of the bands with masses of 8920, 9420, and 9720 Da of the sdLDL from patients compared with the controls was observed. All these three bands were purified by 1-D gels and electro elution, followed by SELDI analysis, and subsequently identified by their matched peptide ions by MS/MS analysis as human apoCIII. The differences in molecular weight of the three polymorphs suggest that they represent different states of sialidation (Pullinger et al., 1997). FIG. 3 demonstrates the profiles of patients and controls from study 2 in the molecular weight range between 3000 to 8000 Da analysed on the CM10 protein chip arrays using SELDI. The two bands at 6420 and 6620 Da were clearly more prominent in the sdLDL of controls than in that of patients. These two bands were also purified by 1-D gel analysis and electro elution, followed by SELDI and then identified as apoCI by immunoblotting. The two bands of apoCI probably represent different states of glycation. Also, SELDI analysis of mass region between 10 000 to 50 000 Da allowed the identification and evaluation of apoAI and apoE in the two LDL subclasses. ApoA1 and apoE were most prominent in the sdLDL of controls compared to that of patients (data not shown).

Table 3 shows the relative content of the identified apolipoproteins in the sdLDL (fraction 5) after correction for apoB100 content in both studies. The sdLDL from the patients has significant higher content of the three isoforms of apoCIII, when compared with the equivalent dense fraction from the matched controls in study 1. On the other hand, the patients showed a significantly lower content of apoCI, apoAI and apoE in its sdLDL class than the controls. In study 2 where the LDL subclasses of patients with type 2 diabetes and matched controls also were analysed the sdLDL contained also significantly more of the three isoforms of apoCIII and lower contents of apoCI, apoAI and apoE than the dense fraction of the matched controls.

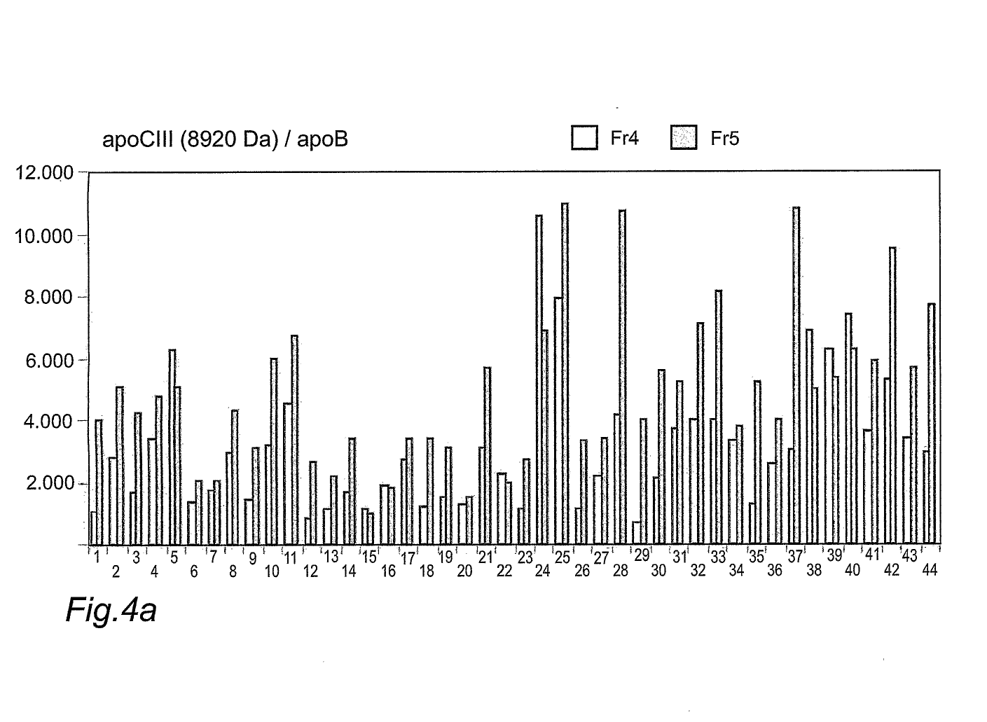

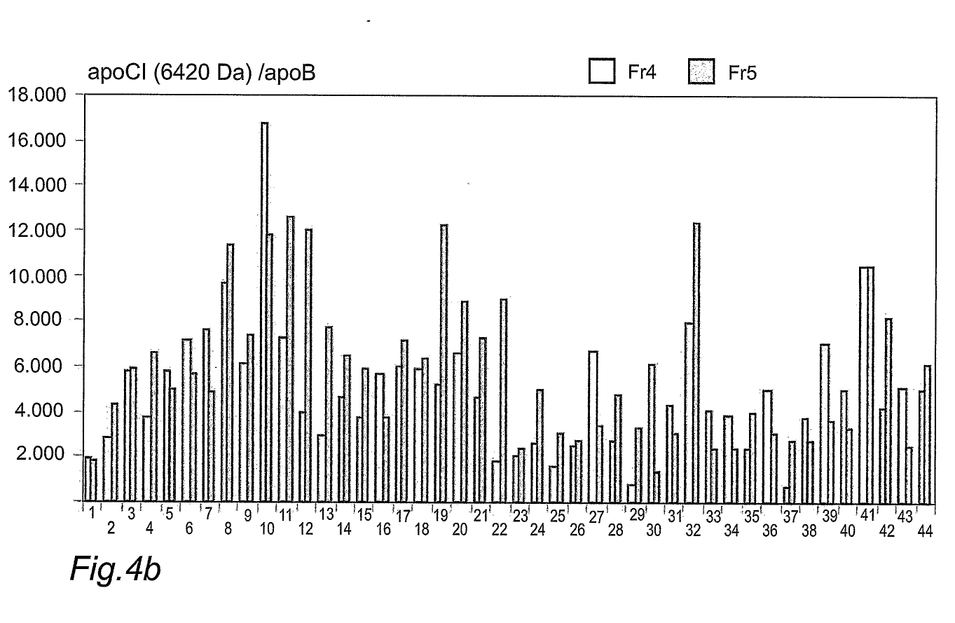

Furthermore, in the controls, there was a significantly higher content of the apoCIII and apoCI in the sdLDL than in the buoyant particles (FIG. 4). In the patients, also the sdLDL had higher content of apoCIII that the buoyant LDL but not of apoCI (FIG. 4).

TABLE 3
Exchangeable apolipoproteins in sdLDL (Fr 5) of patients
with LDL phenotype B and matched controls in both studies

| | STUDY 1 | | STUDY 2 | |
|---|---|---|---|---|
| Apolipo- | Controls | Patients | Controls | Patients |
| protein | (n = 10) | (n = 10) | (n = 23) | (n = 22) |
| CIII-1 | 4.29 ± 2.76 | 6.89 ± 4.71*** | 3.48 ± 1.54 | 6.40 ± 2.43*** |
| (8920) | | | | |
| CIII-2 | 7.30 ± 5.16 | 11.84 ± 6.14*** | 6.23 ± 2.78 | 11.00 ± 4.19*** |
| (9420) | | | | |
| CIII-3 | 3.83 ± 2.84 | 5.38 ± 2.09*** | 3.36 ± 1.22 | 5.28 ± 1.87*** |
| (9720) | | | | |
| CI-1 | 7.57 ± 1.46 | 6.64 ± 2.76* | 7.24 ± 3.09 | 4.30 ± 2.79** |
| (6430) | | | | |
| CI-2 | 6.20 ± 1.59 | 4.57 ± 1.65* | 11.33 ± 5.66 | 6.95 ± 4.64** |
| (6630) | | | | |
| AI | 0.40 ± 0.44 | 0.29 ± 0.26* | 1.22 ± 1.29 | 0.57 ± 0.58** |
| (28130) | | | | |
| E | 0.85 ± 0.59 | 0.42 ± 0.35* | 1.49 ± 0.64 | 1.13 ± 0.53* |
| (33570) | | | | |

Values are means±standard deviation in intensity arbitrary units corrected by apoB100 content of small dense LDL in patients and controls from both studies. Values in parenthesis are molecular weights in Da. The statistical significance level (p) of the differences in apolipoprotein content between the dense LDL class fraction (fr 5) and the same fraction from controls was evaluated with the t-test. Significance levels *p<0.05, **p<0.01, ***p<0.001

### Example 4

### Purification of Differentially Expressed Protein Peaks

Aliquots of LDL fraction 5 from subjects with the B phenotype were pooled and concentrated by vacuum centrifugation, dissolved in 200 μL NuPAGE sample buffer (0.14 M tris, 0.10 M tris-HCL, 0.4 mM EDTA pH 8.5 containing 10% glycerol, 2% LDL, 3% DTT), boiled for 3 min and then separated by the NuPAGE system (Novex (precast gels), San Diego, Calif., USA) using 4-12% Bis-Tris gels (1 well). The NuPAGE (2-N-morpholino) ethane sulfonic acid) MES buffer system (1 M MES, 1 M tris, 69 mM SDS, 20 mM EDTA) was used as running buffer. The mini whole gel eluter (Bio-Rad) was used for electro elution following the manufacturer's instruction. An elution buffer (25 mM histidine, 30 mM 3-(N-morpholino) propane sulphonic acid (MOPS), pH 6.5) was used and the elution was performed at 100 mA for 30 min. Fourteen fractions of approximately 0.5 mL were harvested, and aliquots of 250 μL/fraction was concentrated and analysed by the NUPAGE system followed by SYPRO Ruby staining for subsequent identification of protein bands with MS. The remaining part of the gel eluter fractions was mixed with ice-cold ethanol in 1:4 v/v ratios, precipitated at −20° C. for 2 hours, centrifuged at 10 000×g for 10 min at 4° C., dissolved in 10 μL of 25 mM NH₄HCO₃ and then analysed on NP20 protein chip arrays in order to follow the purification strategy by SELDI analysis.

### Example 5

### MS Analysis of LDL Bound Proteins

### apoCIII, apoAI, apoE

For subsequent MS analysis, the bands detected in the 1-gels were trypsinated and analysed by MS as previously described (Björhall K, Proteomics 2004; 5(1):307-317). Briefly the gel pieces were digested by sequencing grade modified trypsin (Promega, Madison, Wis., USA) and the peptides were extracted with formic acid and acetonitrile. To increase peptide ion signals in MS mode and enable MS/MS analysis, desalting and concentration was carried out using POROS 20-resin (Perseptive Biosystems, Framingham, Mass., USA) following the manufacturer's instruction. The peptides were eluted with 2 μL 70% ACN, 0.1% TFA directly onto a MALDI 100-positions target plate. The spots were allowed to dry prior to application of 1.25 μL of matrix solution CHCA (Agilent Technologies, Waldbronn, Germany diluted 1:1 with 50% acetonitrile, 0.1% trifluofoacetic acid and a final concentration of 0.2 mg/ml diammoniumcitrate). Analysis was then performed on an Applied Biosystem 4700 Proteomics Analyzer MALDI-TOF/TOF mass spectrometer (AB, Framingham, Mass., USA), in reflector mode. MS and MS/MS data analysis was performed using the GPS Explorer™ Software (Applied Biosystems, Framingham, Mass., USA), which utilizes the Mascot peptide mass fingerprinting and MS/MS ion search software (Matrix sciences, London, UK). Identification was considered positive at a confidence level of 95%. The peaks with the masses of 8920, 9420, and 9720 Da were identified as apoCIII after purification with 1-D gels and electro-elution and subsequent identification of their matched peptide ions as MS/MS analysis. The purification procedure was followed by SELDI analysis. The peaks at 28130 and 33570 Da were identified as apoA1 and apoE respectively with the sequence of steps described as for apoCIII.

### Example 6

### Immunoblotting Analysis of apoCI

After 1-D gel analysis of the electroeluter fraction containing the bands at 6420 and 6620 Da respectively analysed by SELDI, the proteins were transferred from the gel onto a PVDF membrane (Millipore, Bedford, Mass., USA) using the semi-dry blotting technique. The membrane was incubated with an antibody against apoC1 (Biosciences), diluted 1:2000 (0.02 μg/mL). For the immunoblotting procedure, the Western Breeze kit (Invitrogen) was used. The bands showed apoC1 immunoreactivity.

### Example 7

### Binding of LDL to Arterial Proteoglycans

Binding of serum LDL to proteoglycans was performed as described using purified versican isolated from swine aortic intima-media with minor modifications (Lindén T, et al, Eur J Clin Invest 1989; 19:38-44). In brief, 50 μl of serum was added to 1.0 ml of buffer A containing 10 μg/ml hexuronate versican, 20 mM NaCl, 10 mM Ca Cl₂, 2 mM MgCl₂, and 5 mM HEPES buffer pH 7.0. Serum was also added to a blank tube containing buffer A without versican. The tubes were incubated at 4° C. for 1 hour, centrifuged 10 min at 12000×g at 4° C. and the pellet washed with 1.0 ml buffer A and the supernatant discarded. The final pellet was dissolved in 100 μl buffer B containing 140 mM NaCl, 5 mM Na₂-EDTA and 10 mM Tris base, pH 10.5. Aliquots of the solution were used for cholesterol determination and for SELDI analysis of the exchangeable apolipoproteins associated with the LDL precipitated from serum by the proteoglycan.

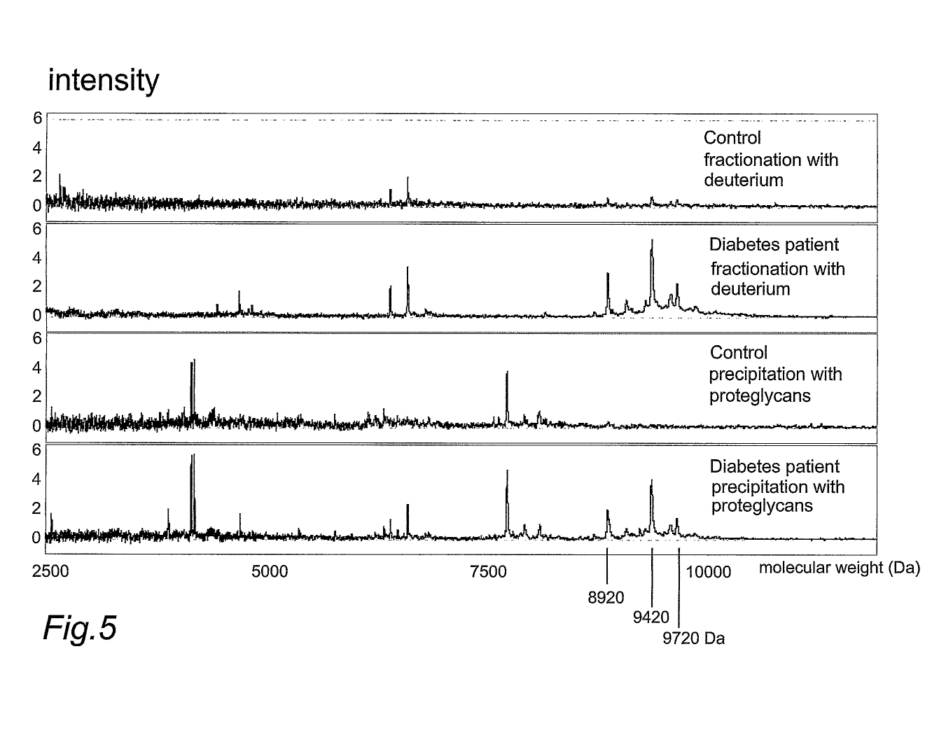

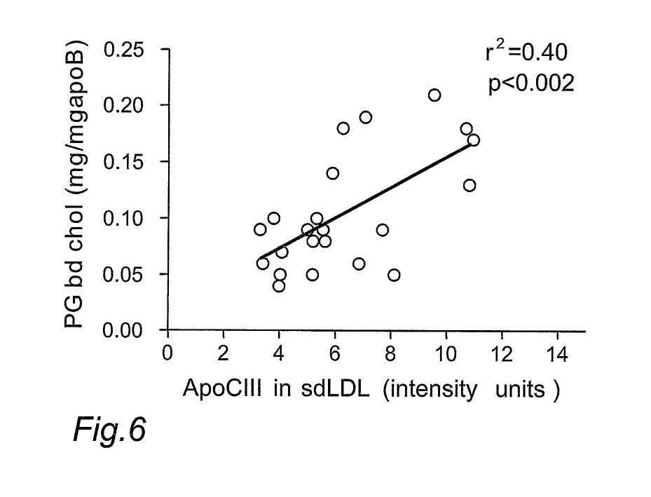

Incubation of serum with arterial versican performed as described causes the precipitation of mainly LDL (Linden T, et al, Eur J Clin Invest 1989; 19:38-44). When corrected for the apoB100 differences the amount of proteoglycan-bound LDL cholesterol (PG-LDL) of the patients sera from study 2 was approximately 32% higher that from controls sera as expected, 115±15 μg of PG-bound cholesterol/mg apoB for the patients vs 80.0±9, in controls, p<0.05. Furthermore, SELDI analyses of the PG-LDL complexes indicated that the insolubilized LDL from patients contained 57, 65, and 58% more of apoCIII-1, apoCIII-2 and apoCIII-3 respectively than the complexes from controls, for example 0.70±0.38 arbitrary units of apoCIII-3 in patients vs 0.30±0.10, in controls, p<0.001 (FIG. 5). The levels of apoCI and apoE respectively (p<0.05) were significantly altered between patients and controls. No statistically significant differences were found in the levels of apoAI. In line with these findings, a significant correlation was found between the total apoCIII content of sdLDL from patients in study 2 and the amount of LDL cholesterol complexed by the PG from serum (FIG. 6). There was no significant correlation between the apoCIII content in sdLDL of controls and the LDL cholesterol insolubilized by the arterial versican.

### Example 8

### Association Between Proteins Bound to sdLDL, Adjusted for apoB Levels, and Cardiovascular Risk Factors and Atherosclerosis

After having verified the hypothesis of a difference in exchangeable apolipoproteins in sdLDL, the aim was to examine whether this composition of proteins is associated with cardiovascular risk factors, inflammation, and subclinical atherosclerosis. All statistics were analysed by using SPSS for Windows 10.0 (SPSS, Inc., Chicago, Ill., USA). The results are presented as numbers, (%), mean values and SD.

ApoC-III was consistently and positively associated to traditional risk factors as well as to markers of inflammation and matrix metalloproteinase-9 in serum. ApoA-I, as well as apoE, and apoC-I showed an inverse relationship to the above-mentioned markers (Table 4).

ApoCIII was positively associated with intima media thickness in the common carotid artery (r=0.35, p=0.02). ApoCI, apoE and apoA1 were inversely associated with mean IMT in the femoral artery (−0.34, p=0.03), the carotid artery bulb (r=−0.43, p=0.01) and the common carotid artery (r=−0.38, p=0.01), respectively. The ratio of apoCIII/apoCI was significantly associated with IMT in the carotid artery bulb and the femoral artery (r=0.31, p=0.049; r=0.38, p=0.02, respectively). In addition the ratio also tended to be associated with IMT in the common carotid artery (r=0.29, p=0.06).

TABLE 4
Relationships between proteins bound to small dense LDL (fr
5), adjusted for apoB in fr 5, and cardiovascular risk factors,
illustrated by Spearman's correlation coefficient.

| | ApoC-III | ApoA-I | ApoE | ApoC-I |
|---|---|---|---|---|
| ApoC-III (plasma) | 0.52 | −0.31 | −0.31 | |
| ApoA-I (plasma) | | 0.33 | 0.56 | |
| SBP | 0.58 | −0.37 | | −0.31 |
| WHR | 0.57 | −0.38 | | −0.42 |
| fB-glucose | 0.38 | | | |
| Pro-insulin | 0.57 | −0.40 | | −0.44 |
| Triglycerides | 0.71 | −0.69 | −0.39 | −0.32 |
| HDL | −0.47 | 0.63 | 0.58 | 0.31 |
| LDL | | | −0.46 | |
| OxLDL | 0.43 | −0.46 | −0.48 | |
| Fibrinogen | | | | −0.40 |
| CRP | 0.52 | | −0.30 | −0.39 |
| IL-18 | 0.45 | | | |
| MMP-9 | 0.32 | −0.49 | −0.33 | −0.38 |

### Example 9

### Odds Ratio for Having Diabetes by Median of apoAI, apoCI, apoCIII or apoE in sdLDL and apoA-1 and apoC-III in Plasma

The concentrations of proteins bound to small dense LDL were adjusted for apoB in fraction 5. In univariate analyses, apo A-I, and apoC-III in plasma, as well as apoCIII, apoA-I, and apoC-I in fraction 5 were all significantly associated with risk of being a diabetes patient (Table 5). ApoE did not show a statistically significant association to diabetes. Since both plasma levels of apoC-III and LDL bound levels of apoC-III were associated with the risk of having diabetes, both these variables were included in a multiple logistic regression analysis. The result of this analysis showed that only apoC-III in sdLDL was an independent predictor of diabetes (Table 6). When including all proteins bound to sdLDL that were univariately associated with risk of having diabetes in the same multivariate logistic regression model, apoC-III and apoC-I (reversely) turned out to be independent predictors of diabetes (Table 7).

Without any further information about clinical characteristics of patients and controls, apoC-III bound to sdLDL above and below median, respectively, correctly classified 76% of the patients and 74% of the controls as having diabetes or not. Similar figures were also obtained for low and high apoC-I (data not shown).

TABLE 5
Univariate odds ratio for having diabetes in subjects with
high and low levels (above and below median, respectively).

| | Odds Ratio | P-value |
|---|---|---|
| ApoCIII plasma | 3.8 | 0.038 |
| ApoCIII fr 5 | 9.0 | 0.002 |
| ApoA1 plasma | 0.16 | 0.009 |
| ApoA1 fr 5 | 0.27 | 0.038 |
| ApoE fr 5 | 0.40 | 0.135 |
| ApoC1 fr 5 | 0.11 | 0.002 |

Values for small dense LDL associated proteins (fr 5) are adjusted for apoB in fr 5.

TABLE 6
Multivariate odds ratio for having diabetes in subjects
with high and low (above and below median, respectively)
values of apoCIII in plasma and on small dense LDL.

| | Odds Ratio | P-value |
|---|---|---|
| ApoCIII plasma | 2.2 | 0.270 |
| ApoCIII fr 5 | 7.3 | 0.006 |

The value for small dense LDL associated apoC-III (fr 5) is adjusted for apoB in fr 5.

TABLE 7
Multivariate odds ratio for having diabetes in subjects
with high and low (above and below median, respectively)
values of small dense LDL associated protiens.

| | Odds Ratio | P-value |
|---|---|---|
| ApoCIII fr 5 | 10.4 | 0.009 |
| ApoA1 fr 5 | 0.772 | >0.300 |
| ApoC1 fr 5 | 0.09 | 0.008 |
| ApoE fr 5 | 0.603 | >0.300 |

Values for small dense LDL associated proteins (fr 5) are adjusted for apoB in fr 5.

1. A method for diagnosing or prognosing susceptibility to develop type 2 diabetes, metabolic syndrome, sub clinical atherosclerosis, myocardial infarct, stroke or clinical manifestations of diabetes in a subject, comprising detecting and quantifying the amount of bound protein(s) on small dense low density lipoproteins (sdLDL) particles in a blood sample from said subject.

2. A method according to claim 1, wherein the diagnosing or prognosing susceptibility is determined of the subject according to the amount and type of proteins bound to sdLDL.

3. A method according to claim 1, wherein said method is performed on serum separated from said blood sample.

4. A method according to claim 3, wherein a fraction containing sdLDL particles is separated from said serum.

5. A method according to claim 1, wherein the amount of bound protein(s) on the sdLDL particles is compared to quantified amounts from healthy control subjects.

6. A method according to claim 1, wherein the detection and quantification of the amounts of bound protein(s) on the sdLDL particles is performed by means of Surface Enhanced Laser Desorption Ionization (SELDI) analysis.

7. A method according to claim 1, wherein the sdLDL particles are isolated from blood or serum by running a density gradient ultra centrifugation.

8. A method according to claim 7, wherein the density gradient ultracentrifugation is carried out with buffers prepared in D₂O.

9. A method according to claim 3, wherein the sdLDL particles are separated by means of precipitation of the sdLDL particles from the serum with an arterial proteoglycan solution.

10. A method according to claim 1, wherein said bound protein is selected from the group consisting of: apolipoprotein CIII, apolipoprotein CI, apolipoprotein A1 and apolipoprotein E.

11. A method according to claim 5, wherein an elevated amount of one or more bound proteins to sdLDL particles relative to that in control subjects is indicative of whether the subject is suffering from diabetes type 2.

12. A method according to claim 5, wherein a lowered amount of one or more bound proteins to sdLDL particles relative to that in control subjects is indicative of whether the subject is suffering from diabetes type 2.

13. A method according to claim 10, wherein the amount of apolipoprotein CI and/or apolipoprotein A1 and/or apolipoprotein E bound to sdLDL particles is lowered and the amount of apoCIII bound to sdLDL particles is elevated as compared to healthy control subjects.

14-19. (canceled)

20. A kit for diagnosing or prognosing susceptibility to develop type 2 diabetes, metabolic syndrome, sub clinical atherosclerosis, myocardial infarct, stroke or clinical manifestations of diabetes in a subject, comprising a protein chip capable of detecting and quantifying proteins bound on sdLDL particles.

21. A kit according to claim 20, wherein said chip is provided with coupled arterial proteoglycans.

22. A kit according to claim 20, wherein said chip directly determines sdLDL bound proteins.

23. A method to predict arterial intima media thickness, comprising detecting and quantifying the amount of bound protein(s) on small dense low density lipoproteins (sdLDL) particles in a blood sample from said subject.
